(19) United States
(12) Patent Application Publication
(10) Pub. No.: US 2018/0024149 A1
(43) Pub. Date: Jan. 25, 2018

(54) PREDICTIVE MARKERS OF IBD

(71) Applicant: NESTEC S.A., Vevey (CH)

(72) Inventors: Eduardo Schiffrin, Crissier (CH); Viral Brahmbhatt, Guragon, Haryana (IN); Francois-Pierre Martin, Vuisternens-devant-Romont (CH); Ivan Montoliu Roura, Lausanne 26 (CH); Serge Andre Dominique Rezzi, Semsales (CH); Jalil Benyacoub, Epalinges (CH)

(21) Appl. No.: 15/544,158

(22) PCT Filed: Jan. 22, 2016
(86) PCT No.: PCT/EP2016/051286

§ 371 Date: Jul. 17, 2017

(30) Foreign Application Priority Data
Jan. 23, 2015 (EP) 15152325.5

(51) Int. Cl. G01N 33/92; G01N 33/53; G01N 33/68

(52) U.S. Cl. CPC G01N 33/92; G01N 33/6893; G01N 33/53; G01N 2800/065; G01N 2800/54; G01N 2405/04; G01N 2405/08

(57) ABSTRACT

The invention relates to a method, a system and kit for identifying the sample of a patient having IBD or predicting the relapse of IBD in patients based on the measurement of biomarkers in a sample of the patient. The biomarkers can be lipids or amino acids or a combination thereof.

### BACKGROUND

Inflammatory bowel disease (IBD) is a group of inflammatory conditions of the colon and small intestine. The disease may cause severe abdominal pain and nutritional problems. The major types of IBD are Crohn's disease (CD) and ulcerative colitis (UC). CD and UC mainly differ by their location and nature of the inflammatory changes. CD can affect any part of the gastrointestinal tract, from the oral cavity to the anus. UC is restricted to the colon and the rectum.

The factors responsible for the disease have not yet been successfully determined. It is expected that genetic background, environmental factors and immunological disorders attribute to the disease.

It is presently unclear how nutritional intake is connected to the disease. The body metabolizes nutritional ingredients to smaller units, the metabolites. These metabolites may have a direct or indirect effect on the presence of IBD. Therefore, there might be a link between the nature and the concentration of the metabolites and the presence and status of the disease.

Thus, dietary habits are considered to be a very important environment factor. It is therefore speculated that nutritional intake may be responsible for inducing, avoiding or potentially treating the disease. Mixtures of prebiotics and probiotics have been used to successfully treat the disease. Beattie et al (1994; Aliment. Pharmacol. Ther.; 8: 1-6) have reported the use of the acid casein fraction in an infant formula in the treatment of 7 children with active small bowel Crohn's disease. U.S. Pat. No. 5,952,295 describes the use of a casein fraction rich in TGF-beta2 for the treatment or prophylaxis of inflammatory conditions of the gastro-intestinal tract, in particular IBD.

Presently, anti-inflammatory drugs, like corticosteroid drugs or mesalazine, antibiotics and, in very severe cases surgery are the preferred choices for treating IBC. While in most cases the above described drugs can already induce remission of the symptoms of IBD an acute resurgence/relapse of the symptoms can appear following treatment. It is not yet possible to reliably predict these so-called flare-ups or indicate which patients are prone to relapses. For example, in children, Crohn's disease has a chronic relapsing course in which up to 50% of the patients eventually need surgery (Davies, G et al; 1990; Br. J. Surg.; 77: 81-94).

The natural clinical course of inflammatory bowel disease (IBD) is characterized by episodes of relapse and remission. The main treatment goal in IBD is to induce and maintain remission by effective control of the gut inflammatory process. Despite the existence of effective treatments for induction of remission of the disease, the assessment of the level of resolution of the inflammatory process at the intestinal level remains uncertain in the current clinical practice. Subclinical inflammation may still persist at the end of a therapeutic cycle that otherwise can be considered successful from a clinical point of view. An “incomplete” biological remission of the inflammatory process is supposed to represent a higher risk for earlier relapse.

In the present application we describe alterations in concentrations of serological metabolites which will predict the likelihood of a relapse prior to its occurrence during the remission period. Hence, this creates a possibility of an early pharmacological/nutritional intervention in this patient population to maintain the state of remission.

Serum laboratory and fecal markers have been proposed for the subclinical detection of disease activity and the prediction of short- or medium term relapses. Calprotectin and high sensitivity C-reactive protein (hs-CRP) have been explored in their capacity to predict disease relapses with a reasonable sensitivity and specificity but they have not been yet entirely accepted in the medical practice. Indeed the cutoff values of the mentioned biomarkers for distinguishing active and inactive disease are not fully defined. For hs-CRP some studies propose a cutoff value of 10 mg/L but this value is far from been accepted by most of the groups. The predictive power of hs-CRP to predict clinical relapse during the follow up of patients is limited.

Thus, it is desirable to identify markers which can be used as diagnostic tools for IBD and to use those markers to evaluate the effectiveness of nutritional interventions.

EP 2330219 generally describes methods for identifying small molecules from biological samples and methods for treating patients with abnormal small molecule profiles. However, the publication does not provide a connection between particular metabolites and particular diseases. WO2010045180 is directed to biomarkers for IBD and methods using the same. However, the publication relates to a very high number of potential biomarkers and also does not specify whether the serum concentration of a particular biomarker is negatively or positively correlated with IBD status.

### SUMMARY OF THE INVENTION

The invention is directed to an analytical method of determining whether a subject has inflammatory bowel disease (IBD) or is prone to a relapse of IBD, providing an isolated biological sample, measuring the concentration of a biomarker in the sample, wherein the concentration is lower or higher in comparison to the reference value for said biomarker indicates that the subject has inflammatory bowel disease, characterized in that the biomarker is a lipid selected from the group consisting of a glycerophospholipid, particularly a phosphatidylcholine, and a sphingolipid or combinations thereof. At least one further biomarker is selected from the group of amino acids consisting of arginine, glutamine, glycine, histidine, isoleucine, leucine, ornithine, serine, threonine, tryptophane, tyrosine phenylalanine, and valine or any combination thereof.

In a preferred embodiment the analytical method of determining whether a subject has inflammatory bowel disease (IBD) or is prone to a relapse of IBD, comprises providing an isolated biological sample, measuring the concentration of a biomarker in the sample, wherein the concentration is lower or significantly lower in comparison to the reference value for said biomarker indicates that the subject has inflammatory bowel disease, characterized in that the biomarker is a lipid selected from the group consisting of a glycerophospholipid, particularly a phosphatidylcholine, and a sphingolipid or combinations thereof.

The phosphatidylcholine can be selected from the group consisting of monoacylphosphatidylcholine, diacylphosphatidylcholine, and alkylacylphosphatidylcholine. The sphingolipid can be a sphingomyelin.

In a preferred embodiment, at least one further biomarker is measured.

The at least one further biomarker is preferably selected from the group of amino acids consisting of arginine, glutamine, glycine, histidine, isoleucine, leucine, ornithine, serine, threonine, tryptophane, tyrosine phenylalanine, and valine or any combination thereof.

The at least one further biomarker can be selected from the group of amino acids consisting of arginine, glycine, histidine, serine, tryptophan, and phenylalanine or any combination thereof.

The at least one further biomarker can be arginine combined with at least one further biomarker being selected from the group of amino acids consisting of glycine, histidine, serine, tryptophan, and phenylalanine or any combination thereof.

The at least one further biomarker can be glycine combined with least one further biomarker being selected from the group of amino acids consisting of arginine, histidine, serine, tryptophan, and phenylalanine or any combination thereof.

The at least one further biomarker can be histidine combined with at least one further biomarker being selected from the group of amino acids consisting of arginine, glycine, serine, tryptophan, and phenylalanine or any combination thereof.

The at least one further biomarker can be serine combined with at least one further biomarker being selected from the group of amino acids consisting of arginine, glycine, histidine, tryptophan, and phenylalanine or any combination thereof.

The at least one further biomarker can be tryptophan combined with at least one further biomarker being selected from the group of amino acids consisting of arginine, glycine, histidine, serine, and phenylalanine or any combination thereof.

The at least one further biomarker can be phenylalanine combined with at least one further biomarker being selected from the group of amino acids consisting of arginine, glycine, histidine, serine, and tryptophan or any combination thereof.

The at least one further biomarker can be selected from the group consisting of arginine, glutamine, isoleucine, serine, and threonine or any combination thereof.

In a preferred embodiment the analytical method of determining whether a subject has inflammatory bowel disease (IBD) or is prone to a relapse of IBD, comprises providing an isolated biological sample, measuring the concentration of at least two biomarkers in the sample, wherein the concentration is lower or significantly lower in comparison to the reference value for a said biomarker indicates that the subject has inflammatory bowel disease, characterized in that a first biomarker is a lipid selected from the group consisting of a glycerophospholipid, particularly a phosphatidylcholine, and a sphingolipid or combinations thereof, and at least one further biomarker is an amino acid selected from the group of amino acids consisting of arginine, glutamine, glycine, histidine, isoleucine, leucine, ornithine, serine, threonine, tryptophane, tyrosine phenylalanine, and valine or any combination thereof.

The phosphatidylcholine can be selected from the group consisting of monoacylphosphatidylcholine, diacylphosphatidylcholine, and alkylacylphosphatidylcholine. The sphingolipid can be a sphingomyelin.

The at least one further biomarker can be selected from the group of amino acids consisting of arginine, glycine, histidine, serine, tryptophan, and phenylalanine or any combination thereof.

The at least one further biomarker can be selected from the group consisting of arginine, glutamine, isoleucine, serine, and threonine or any combination thereof.

The at least one further biomarker can be arginine combined with at least one further biomarker being selected from the group of amino acids consisting of glycine, histidine, serine, tryptophan, and phenylalanine or any combination thereof.

The at least one further biomarker can be glycine combined with least one further biomarker being selected from the group of amino acids consisting of arginine, histidine, serine, tryptophan, and phenylalanine or any combination thereof.

The at least one further biomarker can be histidine combined with at least one further biomarker being selected from the group of amino acids consisting of arginine, glycine, serine, tryptophan, and phenylalanine or any combination thereof.

The at least one further biomarker can be serine combined with at least one further biomarker being selected from the group of amino acids consisting of arginine, glycine, histidine, tryptophan, and phenylalanine or any combination thereof.

The at least one further biomarker can be tryptophan combined with at least one further biomarker being selected from the group of amino acids consisting of arginine, glycine, histidine, serine, and phenylalanine or any combination thereof.

The at least one further biomarker can be phenylalanine combined with at least one further biomarker being selected from the group of amino acids consisting of arginine, glycine, histidine, serine, and tryptophan or any combination thereof.

At least the concentration of 2, 3, 4 or the concentration of 5 biomarkers can be measured.

Each of the biomarkers can be measured in a separate tissue wherein the sample can be selected from the group consisting of whole blood, blood serum, or plasma, tissue of the intestinal wall, tissue of the liver, or mesenteric adipose.

The patient can be a pediatric patient, an adult patient, a patient in a non-acute phase of IBD, or a patient in an acute phase of IBD.

A concentration of the biomarker in the sample of the subject that is significantly lower, or significantly higher, than a reference value for said biomarker indicates that the subject has increased risk of having an IBD relapse.

The concentration of the respective biomarker can be lower or higher than the reference value by 10%, 20%, 30%, 40% or 50% and indicate that the subject is in the acute phase of IBD or is at an increased risk of relapse.

The invention is also directed to the biomarkers or combination of biomarkers as defined above for identifying the samples of subjects in need of an IBD intervention.

The invention is also directed to a system for determining whether a subject has inflammatory bowel disease, the system comprising means for measuring the concentration of the biomarkers of the invention. The system can comprise a computer. The computer can store a data base comprising reference values for the concentrations of the biomarkers and said computer stores a software program having instructions causing the computer to receive and store the measured values of the parameters of the sample of a subject; to compare values of said measured parameters to the reference values stored in the system; to indicate the sample as belonging to a subject having inflammatory bowel disease if the measured values differ from the reference values; output the results indicating that the sample is belonging to a subject having inflammatory bowel disease if the measured values differ from the reference values.

The invention is also directed to a kit for determining whether a subject has inflammatory bowel disease comprising reagents for measuring the concentration of the biomarkers of the invention.

The reagents can be selected from the group of monoclonal antibodies, polyclonal antibodies, single chain antibodies, F_{c} fragments and wherein the reagents are specific for said biomarkers.

### BRIEF DESCRIPTION OF THE FIGURES

The following key identifies the metabolites represented in the figures:

Val—valine;

xLeu—leucine and isoleucine;

Thr—threonine;

Pro—proline;

Ser—serine;

Arg—arginine;

Gly—glycine;

Gln—glutamine;

Met—methionine;

Orn—Ornithine;

Hist—histidine;

Phe—phenylalanine;

Tyr—tyrosine;

Trp—tryptophane,

PCaa—choline glycerophospholipid;

LysoPC—Lysophosphocholine;

PCae—choline glycerophospholipid with an ether bond;

SM (OH)—hydroxy-Sphingomyelines.

lysoPC.a. C18.1—a compound of molecular weight between 519-523 g/mol and which is a monoacylphosphatidylcholine;

lysoPC.a.C18.2—a compound of molecular weight between 517-521 g/mol and which is a monoacylphosphatidylcholine;

PC.aa.C28.1—a compound of molecular weight between 674-678 g/mol and which is a diacylphosphatidylcholine;

PC.aa.C30.0—a compound of molecular weight between 704-708 g/mol and which is a diacylphosphatidylcholine;

PC.aa.C32.2—a compound of molecular weight between 728-732 g/mol and which is a diacylphosphatidylcholine;

PC.aa.C34.2—a compound of molecular weight between 756-760 g/mol and which is a diacylphosphatidylcholine;

PC.aa.C34.3—a compound of molecular weight between 754-758 g/mol and which is a diacylphosphatidylcholine;

PC.aa.C36.2—a compound of molecular weight between 784-788 g/mol and which is a diacylphosphatidylcholine;

PC.aa.C36.3—a compound of molecular weight between 782-786 g/mol and which is a diacylphosphatidylcholine;

PC.aa.C42.6—a compound of molecular weight between 860-864 g/mol and which is a diacylphosphatidylcholine;

PC.ae.C30.0—a compound of molecular weight between 690-694 g/mol and which is an alkylacylphosphatidylcholine;

PC.ae.C34.2—a compound of molecular weight between 742-746 g/mol and which is an alkylacylphosphatidylcholine;

PC.ae.C36.2—a compound of molecular weight between 770-774 g/mol and which is an alkylacylphosphatidylcholine;

PC.ae.C38.2 —a compound of molecular weight between 798-802 g/mol and which is an alkylacylphosphatidylcholine;

PC.ae.C38.3—a compound of molecular weight between 796-800 g/mol and which is an alkylacylphosphatidylcholine;

SM (OH) C14:1—a compound of molecular weight between 687-691 g/mol and which is a sphingomyelin.

FIG. 1: Metabolic differences between IBD children (crohn's disease exclusively), and non-IBD children observed in blood plasma at fasting stage. Box plots illustrating the concentration levels (ng/100 μL plasma) of representative amino acid and lipid metabolites measured by targeted UHPLC-ESI-MS/MS metabonomic analysis at fasting at the start of the study before any nutritional intervention in CD children (n=12, 9 males, 3 females), and from non-IBD children (n=20, 10 males, 10 females).

Significant differences were assessed by Mann-Whitney U test and marked as follows: *p<0.05., **p<0.01, ***p<0.001.

FIG. 2: Metabolic differences between IBD children (crohn's disease exclusively), and non-IBD children observed in blood plasma at fasting stage.

Box plots illustrating the concentration levels (ng/100 uL plasma) of representative amino acid and lipid metabolites measured by targeted UHPLC-ESI-MS/MS metabonomic analysis at fasting at the start of the study before any nutritional intervention in CD children (n=12, 9 males, 3 females), and from non-IBD children (n=20, 10 males, 10 females).

Significant differences were assessed by Mann-Whitney U test and marked as follows: *p<0.05., **p<0.01, ***p<0.001.

FIG. 3: Metabolic differences between IBD children (crohn's disease exclusively), and non-IBD children observed in blood plasma at fasting stage.

Box plots illustrating the concentration levels (ng/100 uL plasma) of representative amino acid and lipid metabolites measured by targeted UHPLC-ESI-MS/MS metabonomic analysis at fasting at the start of the study before any nutritional intervention in CD children (n=12, 9 males, 3 females), and from non-IBD children (n=20, 10 males, 10 females).

Significant differences were assessed by Mann-Whitney U test and marked as follows: *p<0.05., **p<0.01, ***p<0.001.

FIG. 4: Metabolic differences between IBD children (crohn's disease exclusively), and non-IBD children observed in blood plasma at fasting stage.

Box plots illustrating the concentration levels (ng/100 uL plasma) of representative amino acid and lipid metabolites measured by targeted UHPLC-ESI-MS/MS metabonomic analysis at fasting at the start of the study before any nutritional intervention in CD children (n=12, 9 males, 3 females), and from non-IBD children (n=20, 10 males, 10 females).

Significant differences were assessed by Mann-Whitney U test and marked as follows: *p<0.05., **p<0.01, ***p<0.001.

Definitions

Biomarker means a compound, preferably a metabolite, that is differentially present (i.e., increased or decreased) in a biological sample from a subject or a group of subjects having a first phenotype (e.g., having a disease) as compared to a biological sample from a subject or group of subjects having a second phenotype (e.g., not having the disease). A biomarker may be differentially present at any level, but is generally present at a level that is increased by at least 5%, by at least 10%, by at least 15%, by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%, by at least 100%, by at least 110%, by at least 120%, by at least 130%, by at least 140%, by at least 150%, or more; or is generally present at a level that is decreased by at least 5%, by at least 10%, by at least 15%, by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%, or by 100% (i.e., absent).

Significance is important for assessing the relevance of a difference from a reference value. A biomarker is preferably differentially present at a level that is statistically significant. Thus statistically different means a p-value less than 0.05, less than 0.01, or less than 0.001 and/or a q-value of less than 0.10 as determined using either Mann-Whitney U test, Welch's T-test or Wilcoxon's rank-sum Test).

The level of one or more biomarkers means the absolute or relative amount or concentration of the biomarker in the sample. A level that is lower means a numerical value that is lower than the reference value and does not include the reference value. Sample or biological sample means biological material isolated from a subject. The biological sample may contain any biological material suitable for detecting the desired biomarkers, and may comprise cellular and/or non-cellular material from the subject. The sample can be isolated from any suitable biological tissue or fluid such as, for example, whole blood, blood serum, or plasma, tissue of the intestinal wall, tissue of the liver, or mesenteric adipose stool, blood, blood plasma, blood serum, or urine.

Subject means any animal, but is preferably a mammal, such as, for example, a human, monkey, mouse, or rabbit.

A reference level or reference of a biomarker means a level that is indicative of a lack of a particular disease state or phenotype, in particular, IBD. Thus, these are the levels that are generally found in healthy subjects. A reference or default level will usually be a numerical value and be expressed in the form of a concentration (g/l; ng/μL; ng/100 μL)

Metabolic profile means a complete or partial inventory of small molecules within a targeted cell, tissue, organ, organism, or fraction thereof (e.g., cellular compartment). The inventory may include the quantity and/or type of small molecules present. Particularly preferred are molecules that are metabolized in the biochemical pathways and can serve as building blocks for cells. The “metabolic profile” may be determined using a single technique or multiple different techniques.

Inflammatory bowel disease or IBD refers to diseases which cause inflammation in the digestive tract, including Crohn's disease and ulcerative colitis. The causes of IBD are unknown and symptoms include abdominal cramps, bloody diarrhea, fever and weight loss.

Crohn's disease or CD refers to a chronic inflammatory disorder of the gastrointestinal (GI) tract. It may occur in any portion of the GI tract but is most often found to affect the small intestine and/or colon. Unlike ulcerative colitis, CD can affect the entire thickness of the bowel wall.

Ulcerative colitis or UC refers to a chronic disease marked by inflammation and ulceration of the mucosa (innermost lining) of the colon or large intestine. UC differs from CD in that UC involves only the colon, the inflammation involves the entire rectum extending up the colon in a continuous manner without areas of normal intestine interspersed with diseased areas, and UC affects only the innermost lining of the colon.

A glycerophospholipid is a lipid and relates to any derivative of sn-glycero-3-phosphoric acid that contains at least one O-acyl, or O-alkyl, or O-alk-1′-enyl residue attached to the glycerol moiety and a polar head made of a phosphate. Glycerophospholipids in the sense of the invention are naturally (in mammals, in particular humans) occurring glycerophospholipids and any derivatives thereof. A derivative differs from the original glycerophospholipid in that one moiety (or residue) has been deleted, modified or added to the original glycerophospholipid.

Choline(s) in the sense of the invention comprise(s) the chemical compound choline (2-hydroxy-N,N,N-trimethylethanaminium, also named Bilineurine, (2-Hydroxyethyl)trimethylammonium) as such and derivatives thereof. A choline derivative in the sense of the invention is 2-hydroxy-N,N,N-trimethylethanaminium covalently attached to glycerol or phosphorylated glycerol. The glycerol backbone can be covalently bound to fatty acids. Choline derivatives in the sense of the invention are naturally (in mammals, in particular humans) occurring phospholipids.

Phosphatidylcholines are a subclass of glycerophospholipids that incorporate choline as a headgroup. The phospholipid is composed of a choline head group and glycerophosphoric acid with a variety of acyl- or alkyl-residues.

The phosphatidylcholines can have a total of from 1 to 50 carbon atoms in the acyl residues or have a total from 3 to 50 carbon atoms in the acyl residues and a total of 1 to 8 double bonds in the acyl residues.

A monoacylphosphatidylcholine (also designated lysophosphatidylcholine) is a phosphatidylcholine containing one O-acyl residue.

A diacylphosphatidylcholine is a phosphatidylcholine containing two O-acyl residues.

An alkylacylphosphatidylcholine is a phosphatidylcholine containing one O-acyl and one O-alkyl residue.

Sphingolipids are lipids containing a backbone of sphingoid bases, a set of aliphatic amino alcohols that includes sphingosine. The sphingosine backbone is O-linked to a (usually) charged head group such as ethanolamine, serine, or choline. The backbone is also amide-linked to an acyl group, such as a fatty acid.

Sphingolipids can have a total number of carbon atoms in the acyl chains from 10 to 30 or have a total number of carbon atoms in the acyl chains from 10 to 30 and 1 to 5 double bonds.

Sphingomyelin in the sense of the invention are sphingomyelin as defined in the following way: Sphingomyelin comprises sphingosin (D-Erythro-2-aminooctadec-4-en-1,3-diol). A fatty acid is covalently attached to the C2-aminogroup of the sphingosin via an amid-bond. A phosphate group is covalently attached to the Ci-hydroxyl-group of the sphingosin via a phosphoesther-bond. In addition, sphingomyelin in the sense of the invention also encompasses sphingomyelin derivatives, i.e. means additional residues.

PCaa is a choline glycerophospholipid.

LysoPC is a Lysophosphocholine.

PCae is a choline glycerophospholipid with an ether bond.

SM (OH) is a hydroxy-sphingomyeline. Hydroxysphinogomyelines can have a total number of carbon atoms in the acyl residues from 10 to 30.

IysoPC.a.C18.1 is a compound of molecular weight between 519-523 g/mol and which is a monoacylphosphatidylcholine.

IysoPC.a.C18.2 is a compound of molecular weight between 517-521 g/mol and which is a monoacylphosphatidylcholine.

PC.aa.C28.1 is a compound of molecular weight between 674-678 g/mol and which is a diacylphosphatidylcholine.

PC.aa.C30.0 is a compound of molecular weight between 704-708 g/mol and which is a diacylphosphatidylcholine.

PC.aa.C32.2 is a compound of molecular weight between 728-732 g/mol and which is a diacylphosphatidylcholine.

PC.aa.C34.2 is a compound of molecular weight between 756-760 g/mol and which is a diacylphosphatidylcholine.

PC.aa.C34.3 is a compound of molecular weight between 754-758 g/mol and which is a diacylphosphatidylcholine.

PC.aa.C36.2 is a compound of molecular weight between 784-788 g/mol and which is a diacylphosphatidylcholine.

PC.aa.C36.3 is a compound of molecular weight between 782-786 g/mol and which is a diacylphosphatidylcholine.

PC.aa.C42.6 is a compound of molecular weight between 860-864 g/mol and which is a diacylphosphatidylcholine.

PC.ae.C30.0 is a compound of molecular weight between 690-694 g/mol and which is an alkylacylphosphatidylcholine.

PC.ae.C34.2 is a compound of molecular weight between 742-746 g/mol and which is an alkylacylphosphatidylcholine.

PC.ae.C36.2 is a compound of molecular weight between 770-774 g/mol and which is an alkylacylphosphatidylcholine.

PC.ae.C38.2 is a compound of molecular weight between 798-802 g/mol and which is an alkylacylphosphatidylcholine.

PC.ae.C38.3 is a compound of molecular weight between 796-800 g/mol and which is an alkylacylphosphatidylcholine.

SM (OH) C14:1 is a compound of molecular weight between 687-691 g/mol and which is a sphingomyelin.

### DETAILED DESCRIPTION OF THE INVENTION

The section headings serve to clarify the subject matter and should not be interpreted to limit the subject matter. If ranges of values are disclosed each individual value is considered to be covered by the range, in particular, each integer number. If not noted otherwise, values in % relate to weight/weight (w/v) values.

The inventors have identified a metabolic signature in patients with IBD that is different from age-matched controls. The reduced concentration of these metabolites, in the absence of clinical symptoms, is expected to be due to persistence or reactivation of subclinical inflammation during clinical remission. Thus, reduced concentrations of these metabolites in IBD patients, when compared with age-matched controls, will be predictors of clinical relapse, increased need for close monitoring and/or need for a pharmacological/nutritional intervention to maintain the state of remission. Further, persisting imbalance of the metabolic profile may indicate a patient with a severe profile of disease, i.e. may have a higher relapse rate and/or higher severity of disease symptoms and/or lower induction of clinical remissions.

Measurement Methods

Any suitable method may be used to analyze the biological sample in order to determine the level(s) of the one or more biomarkers in the sample. Suitable methods include chromatography (e.g., HPLC, gas chromatography, liquid chromatography), mass spectrometry (e.g., MS, MS-MS), enzyme-linked immunosorbent assay (ELISA), antibody linkage, other immunochemical techniques, and combinations thereof. Further, the level(s) of the one or more biomarkers may be measured indirectly, for example, by using an assay that measures the level of a compound (or compounds) that correlates with the level of the biomarker(s) that are desired to be measured.

Comparison Methods

The level(s) of the one or more biomarkers may be compared to inflammatory bowel disease reference levels using various techniques, including a simple comparison (e.g., a manual comparison) of the level(s) of the one or more biomarkers in the biological sample to inflammatory bowel disease-positive and/or inflammatory bowel disease-negative reference levels. The level(s) of the one or more biomarkers in the biological sample may also be compared to inflammatory bowel disease reference levels using one or more statistical analyses (e.g., Mann-Whitney U test, t-test, Welch's T-test, Wilcoxon's rank sum test, random forest).

After the level(s) of the one or more, preferably at least two, biomarkers in the sample is/are determined, the level(s) are compared to IBD reference levels in order to predict whether the subject is predisposed to developing inflammatory bowel disease or determine whether the person has IBD. Levels of the one or more, preferably at least two, biomarkers in a sample lower than the reference levels are indicative of the subject being predisposed to developing inflammatory bowel disease. Levels of the one or more, preferably at least two, biomarkers in a sample being not lower than the reference levels are indicative of the subject not having or not being predisposed to developing inflammatory bowel disease.

Biomarkers

The invention is based on the finding that particular biomarkers and particular combinations of biomarkers are suitable for determining IBD or a relapse of IBD. The biomarkers can be lipids or/and amino acids. These biomarkers can be lipids like glycerophospholipids, sphingolipids, and combinations thereof.

Preferably the glycerophospholipid is phosphatidylcholine.

Preferably the biomarker phosphatidylcholine is a biomarker selected from the group consisting of monoacylphosphatidylcholine, diacylphosphatidylcholine, and alkylacylphosphatidylcholine or combinations thereof.

Preferably the phosphatidylcholines/monoacylphosphatidylcholine/diacylphosphatidylcholine/alkylacylphosphatidylcholine have a total of from 1 to 50 carbon atoms in the acyl residues or have a total from 3 to 50 carbon atoms in the acyl residues and a total of 1 to 8 double bonds in the acyl residues.

Preferably the monoacylphosphatidylcholine is selected from the group consisting of lysoPC.a.C18.1 and lysoPC.a.C18.2, or a combination thereof.

Preferably the diacylphosphatidylcholine is selected from the group consisting of PC.aa.C28.1, PC.aa.C30.0, PC.aa.C32.2, PC.aa.C34.2, PC.aa.C34.3, PC.aa.C36.2, PC.aa.C36.3, PC.aa.C42.6, or combinations thereof.

Preferably the alkylacylphosphatidylcholine is selected from the group consisting of PC.ae.C30.0, PC.ae.C34.2, PC.ae.C36.2, PC.ae.C38.2, PC.ae.C38.3, or combinations thereof.

Preferably the sphingolipid is a sphingomyelin. More preferably the sphingomyelin is hydroxy-sphingomyelines, or more particularly, SM (OH) C14:1.

Sphingolipids, in particular sphingomyelines, can have a total number of carbon atoms in the acyl chains from 10 to 30 or have a total number of carbon atoms in the acyl chains from 10 to 30 and 1 to 5 double bonds; hydroxysphinogomyelines can have a total number of carbon atoms in the acyl residues from 10 to 30.

The amino acids can be selected from the group consisting of arginine, glutamine, glycine, histidine, isoleucine, leucine, ornithine, serine, threonine, tryptophane, tyrosine phenylalanine, and valine or any combination thereof.

Preferred amino acids are those where according to the example significant differences of p<0.001 were observed in children suffering from IBD versus age matched controls. Therefore preferred amino acids are being selected from the group of amino acids consisting of arginine, glycine, histidine, serine, tryptophan, and phenylalanine or any combination thereof.

A preferred amino acid is arginine and at least one further biomarker being selected from the group of amino acids consisting of glycine, histidine, serine, tryptophan, and phenylalanine or any combination thereof.

A preferred amino acid is glycine and at least one further biomarker being selected from the group of amino acids consisting of arginine, histidine, serine, tryptophan, and phenylalanine or any combination thereof.

A preferred amino acid is histidine and at least one further biomarker is being selected from the group of amino acids consisting of arginine, glycine, serine, tryptophan, and phenylalanine or any combination thereof.

A preferred amino acid is serine and at least one further biomarker is being selected from the group of amino acids consisting of arginine, glycine, histidine, tryptophan, and phenylalanine or any combination thereof.

A preferred amino acid is tryptophan and at least one further biomarker is being selected from the group of amino acids consisting of arginine, glycine, histidine, serine, and phenylalanine or any combination thereof.

A preferred amino acid is phenylalanine and at least one further biomarker is being selected from the group of amino acids consisting of arginine, glycine, histidine, serine, and tryptophan or any combination thereof.

Particularly preferred are amino acids being selected from the group consisting of arginine, glutamine, isoleucine, serine, and threonine or any combination thereof.

Also preferred is any combination consisting of 2, 3 or 4 of said amino acids and a combination comprising all 5 of said amino acids. Any possible permutation of the amino acids in said combination is considered to be disclosed by this invention.

The biomarkers are preferably a set of biomarkers comprising phospholipids and amino acids.

Determination/Diagnostic method

The method can be an ex vivo analytical method. The invention is directed to a method of determining whether a subject has inflammatory bowel disease (IBD) or indicating that a subject is prone to a relapse of IBD (optionally during a non-acute phase of IBD), providing an isolated biological sample, measuring the concentration of a biomarker in the sample, wherein the concentration is lower, in particular, significantly lower than a reference value for said biomarker indicates that the subject has IBD or is likely to have a relapse of IBD, characterized in that the biomarker is a lipid or an amino acid.

The IBD can be Crohn's disease (CD) or ulcerative colitis (UC).

The lipids can be selected from a group consisting of choline, choline derivatives, sphingomyelin, and sphingomyelin derivatives. Lipids can be choline or sphingomyelin.

Particularly considered are cholines selected from the group consisting of choline glycerophospholipid (PCaa), choline glycerophospholipid with an ether bond (PCae) and lysophosphocholine (LysoPC) and combinations thereof. Also preferred are combinations consisting of two of said lipids wherein any possible combination out of the three lipids is considered. Also considered is a combination consisting of three of said amino acids. It was surprisingly found that relapse of IBD or the presence of IBD can be predicted using said biomarkers.

When performing the above method the incorporation of additional markers into the set of markers can be considered that are not lipid markers. The additional markers can be any metabolite present in the subject. Particularly preferred biomarkers are amino acids or amino acid combinations as described in the section regarding biomarkers.

The accuracy of the method can be improved by measuring the concentration of at least 2, 3, 4 or of 5 biomarkers. In addition the method can comprise the measuring of the concentration of 1-10, 2-9, 3-8, 4-7, 5-6 biomarkers or any value within the range covered by the indicated lower and upper limits.

In particular, the method is a method for predicting a relapse of IBD. A relapse of IBD can occur in a subject that had been diagnosed with IBD previously (by one of the following symptoms: abdominal pain, vomiting, diarrhea, rectal bleeding, severe internal cramps/muscle spasms in the region of the pelvis, weight loss and various associated complaints or diseases like arthritis, pyoderma gangrenosum, and primary sclerosing cholangitis) but presently does not exhibit the symptoms of IBD.

The biomarkers can be measured in one type of sample or several types of a sample. The sample type can be selected from the group consisting of whole blood, blood serum, or plasma, tissue of the intestinal wall, tissue of the liver, or mesenteric adipose. Alternatively, the biomarkers are measured in at least 2, 3, 4, 5, 6, 7, 8, 9 or 10 different types of samples. The use of different samples is advised when a difference of a concentration of a first biomarker indicating IBD or a relapse of IBD is more pronounced in a first type of sample, while the corresponding difference of second or more biomarker is more pronounced in a sample that is different from the first type of sample.

The patients can be human patients. The patients can be patients of age 1-17 (pediatric patients) or of age 18 and above (adult patients, preferably patients until the age of 67).

The patient can be a patient in acute phase of IBD or in a non-acute phase of IBD. In a non-acute phase of IBD the following symptoms are absent: abdominal pain, vomiting, diarrhea, rectal bleeding, severe internal cramps/muscle spasms in the region of the pelvis, weight loss and various associated complaints or diseases like arthritis, pyoderma gangrenosum, and primary sclerosing cholangitis.

The concentration of the respective biomarker can be lower than the concentration of the reference value by 5%, 10%, 20%, 30%, 40%, or 50%. The concentration of the respective biomarker can be lower than the reference value by 5%-50%, 10%-40%, 20%-30% or any combination of the lower and upper limits indicated. The reference value can be a value provided by the scientific literature or a medical institution in the USA, EU, Switzerland or Germany. The reference value can also be determined by determining said values in at least 1, 2, 5, or 10 subjects that are known not to be suffering from IBD and forming the average of the determined values.

The reference value can be determined for each patient group (pediatric, adult, or others) individually.

System

The above described method can also be performed with the help of system or device wherein the method is being performed on a machine.

Thus, the invention is also directed to a system for determining whether a subject has inflammatory bowel disease or predicting a relapse of IBD, the system comprising means for measuring the concentration of the biomarkers set forth above wherein the system is comprising a computer ; said computer stores a data base comprising reference values for the concentrations of the biomarkers; said computer stores a software program having instructions causing the computer; to receive and store the measured values of the parameters of the sample of a subject; to compare values of said measured parameters to the reference values stored in the system; indicate the sample as belonging to a subject having inflammatory bowel disease if the measured values differ from the reference values; output the results of indicating the sample as belonging to a subject having inflammatory bowel disease or as being a subject that is prone to a relapse of IBD if the measured values differ from the reference values.

The biomarkers can be a lipid or an amino acid. Preferably at least two biomarkers are measured. Preferably a first biomarker can be a lipid and at least one further biomarker can be an amino acid.

These lipid biomarkers can be lipids like glycerophospholipids, sphingolipids, and combinations thereof.

Preferably the glycerophospholipid is phosphatidylcholine.

Preferably the biomarker phosphatidylcholine is a biomarker selected from the group consisting of monoacylphosphatidylcholine, diacylphosphatidylcholine, and alkylacylphosphatidylcholine or combinations thereof.

Preferably the monoacylphosphatidylcholine is selected from the group consisting of lysoPC.a.C18.1 and lysoPC.a.C18.2, or a combination thereof.

Preferably the diacylphosphatidylcholine is selected from the group consisting of PC.aa.C28.1, PC.aa.C30.0, PC.aa.C32.2, PC.aa.C34.2, PC.aa.C34.3, PC.aa.C36.2, PC.aa.C36.3, PC.aa.C42.6, or combinations thereof.

Preferably the alkylacylphosphatidylcholine is selected from the group consisting of PC.ae.C30.0, PC.ae.C34.2, PC.ae.C36.2, PC.ae.C38.2, PC.ae.C38.3, or combinations thereof.

Preferably the sphingolipid is a sphingomyelin. More preferably the sphingomyelin is hydroxy-sphingomyelines, or more particularly, SM (OH) C14:1.

Also preferred are combinations consisting of two of said lipids wherein any possible combination out of three, four, five, six or more lipids is considered. Also considered is a combination consisting of three, four or five of said amino acids. It was surprisingly found that relapse of IBD or the presence of IBD can be predicted using said biomarkers.

When performing the above method additional markers can be considered that are not lipid markers. The additional markers can be any metabolite present in the subject. Particularly preferred biomarkers are amino acids or combinations of amino acids as described in the section regarding biomarkers.

The accuracy of the method can be improved by measuring the concentration of at least 2, 3, 4 or the concentration of 5 biomarkers. In addition, the method can comprise the measuring of the concentration of 1-10, 2-9, 3-8, 4-7, or 5-6 biomarkers or any other range covered by the indicated lower and upper limits.

In particular, the method is a method for predicting a relapse of IBD (indicating whether a subject is prone to a relapse of IBD). A relapse of IBD can occur in a subject that had been diagnosed with IBD previously (by one of the following symptoms: abdominal pain, vomiting, diarrhea, rectal bleeding, severe internal cramps/muscle spasms in the region of the pelvis, weight loss and various associated complaints or diseases like arthritis, pyoderma gangrenosum, and primary sclerosing cholangitis).

The biomarkers can be measured in one type of sample being selected from the group consisting of whole blood, blood serum, or plasma, tissue of the intestinal wall, tissue of the liver, or mesenteric adipose. Alternatively, the biomarkers are measured in at least 2, 3, 4, 5, 6, 7, 8, 9 or 10 different types of samples. The use of different samples is advised when a difference of a concentration of a first biomarker indicating IBD or a relapse of IBD is more pronounced in a first type of sample, while the corresponding difference of second or more biomarker is more pronounced in a sample that is different from the first type of sample.

The patients can be human patients. The patients can be patients of age 1-17 (pediatric patients) or of age 18 and above (adult patients, preferably patients until the age of 67).

The patient can be a patient in acute phase of IBD or in a non-acute phase of IBD. In a non-acute phase of IBD the following symptoms are absent: abdominal pain, vomiting, diarrhea, rectal bleeding, severe internal cramps/muscle spasms in the region of the pelvis, weight loss and various associated complaints or diseases like arthritis, pyoderma gangrenosum, and primary sclerosing cholangitis.

The concentration of the respective biomarker can be lower than the reference value by 5%, 10%, 20%, 30%, 40%, or 50%. The concentration of the respective biomarker can be lower than the reference value by 5%-50%, 10%-40%, 20%-30% or any combination of the lower and upper limits indicated. The difference between the reference value and the determined value can be calculated by the program via subtraction. A difference will be determined as significant if one of the following tests selected from the group consisting of Mann-Whitney U test, t-test, Welch's T-test, Wilcoxon's rank sum test, and random forest indicate the difference as significant. The reference value can be a value provided by the scientific literature or a medical institution in the USA, EU, Switzerland or Germany. The reference value can also be determined by the determined said values in at least 1, 2, 5, or 10 subjects that are known not to be suffering from IBD and forming the average of the determined values.

The reference value can be determined for each patient group (pediatric, adult, or others) individually.

The output can be on display, print out or data carrier wherein said carrier can be a local physical device, or be comprised in a further real or virtual computer.

Kits

The invention is also directed to a kit for determining whether a subject has inflammatory bowel disease comprising reagents for measuring the concentration of the biomarkers of the invention.

The reagents can be used for measuring the concentration of a lipid or an amino acid.

Particularly preferred biomarkers are lipids or combinations of lipids as described in the section regarding biomarkers.

The kit can comprise reagents for biomarkers that are not lipid markers. The additional markers can be any metabolite present in the subject. Particularly preferred biomarkers are amino acids or combinations of amino acids as described in the section regarding biomarkers.

The reagents can be selected from the group consisting of monoclonal antibodies, polyclonal antibodies, single chain antibodies, Fc fragments. The reagents are characterized by being specific for said biomarkers.

### EXAMPLES

### Example 1

Inflammatory activation of the intestine depends on the pathological activation of immune cell populations that are expanded in the intestinal lamina propria and actively secreting pro-inflammatory mediators resulting in tissue damage. This local activation of the immune system results in turn in the systemic activation of the inflammatory process as it is shown by the acute phase response mediators produced by the liver and the mesenteric adipose tissue in IBD.

The mentioned immune/inflammatory activation results in increased metabolic needs by the intestinal and systemic immune system, the intestinal wall, the liver and the mesenteric adipose tissue. In addition to the increased metabolic requirements of different tissues activated cells may produce a different pattern of metabolites during active disease. The combination of the specific nutrient deficiencies together with the altered profile of metabolites produced by different tissues represent a global metabolic alteration that has not been well characterized so far in the IBD. In fact the recognition of the global metabolic alteration may become an important biomarker of disease activity and also a predictor of the disease evolution.

In light of the above-mentioned possibility, we assessed the metabolic profile of pediatric patients with IBD. A cohort of pediatric CD patients was prospectively followed for 12 weeks. The patient characteristics and characteristics of age-matched controls are outlined in table1.

TABLE 1
Patient characteristics

| | CD | Controls |
|---|---|---|
| n | 13 (4 females) | 20 (10 females) |
| Age | 13.9 (6.6-17.7) | 13.9 (10.2-8.3-12) |
| Treatment | Modulen IBD | NA |
| PCDAI (T0) | 30 (12.5-62.5) | NA |
| PCDAI (T4) | 8.75 (0-27.5) | NA |
| PCDAI (T12) | 5 (0-25) | NA |

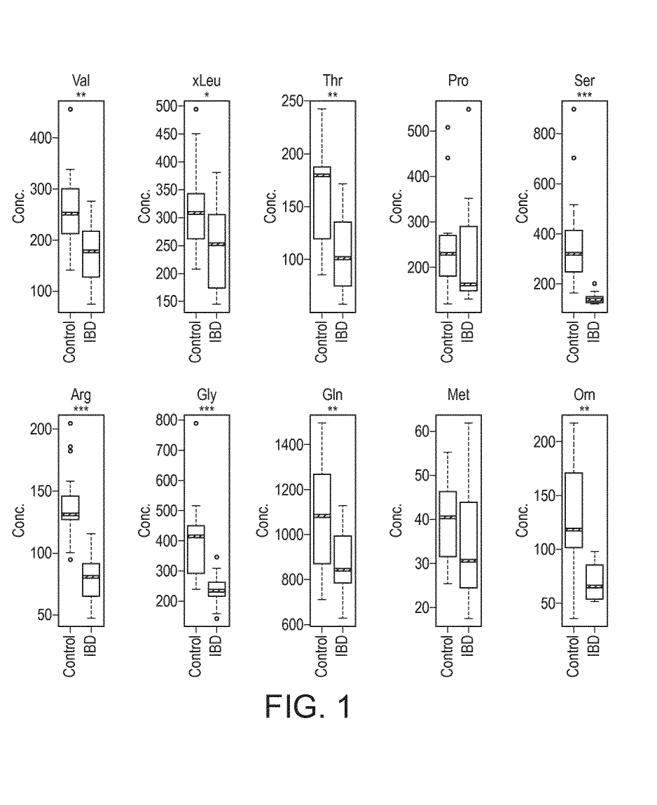

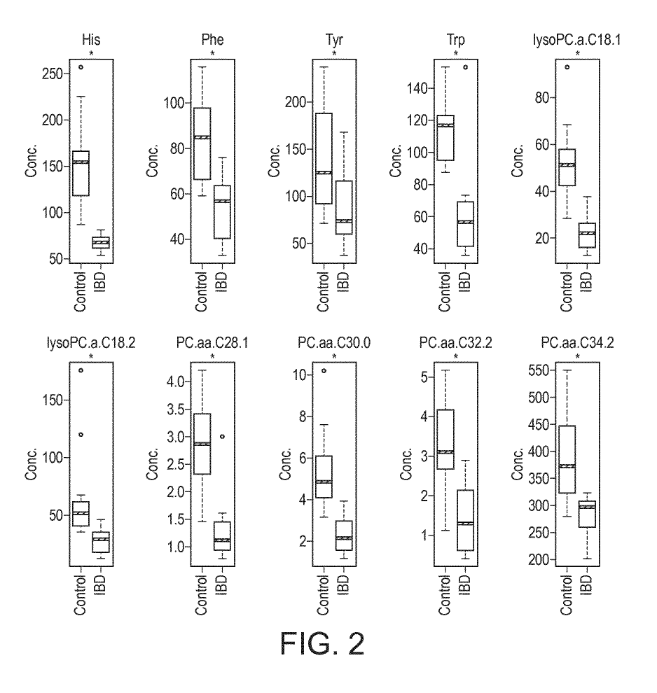

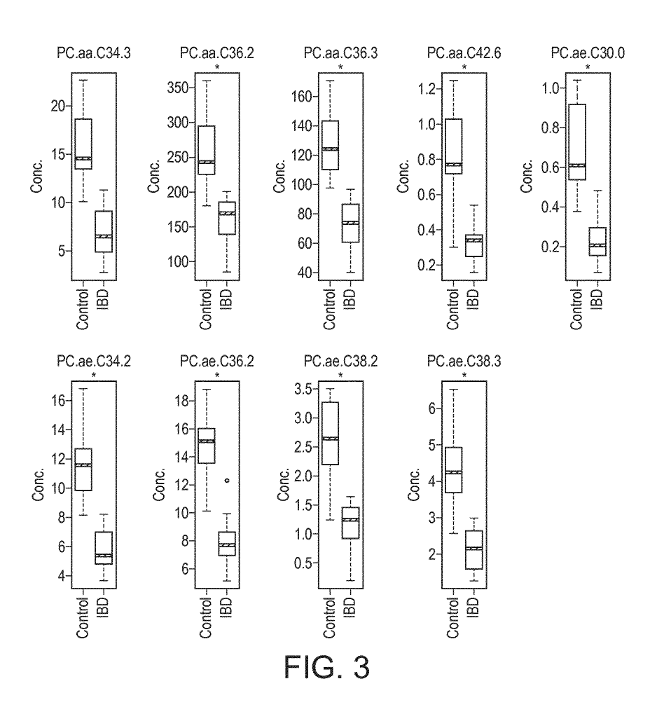

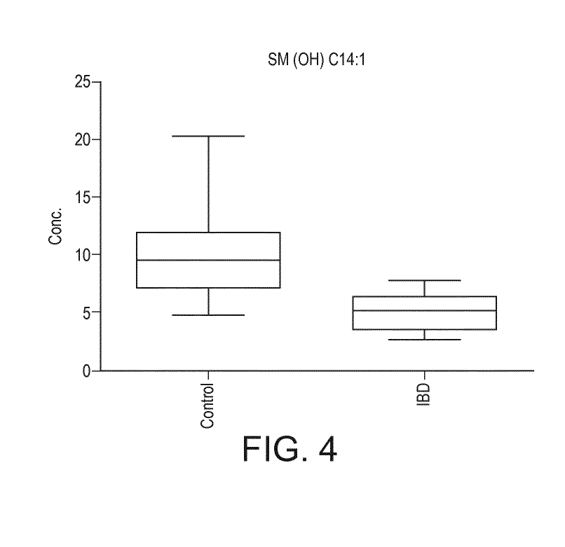

The plasma biochemical composition of IBD patients and age-matched controls was analyzed by metabonomics approach. One method consisted using the Biocrates Life Sciences AbsolutelDQ™ kit according to the manufacturer's instructions. The concentrations of these metabolites were compared with the concentrations detected in non-IBD children of similar age range. Significant differences were assessed by Mann-Whitney U test and results on selected metabolites are displayed in FIGS. 1-4. The metabolic differences demonstrated a clear depletion in circulating concentrations of most amino acids, including valine, leucine, isoleucine, serine, arginine, glycine, glutamine, ornithine, histidine, phenylalanine, tyrosine and tryptophane. This depletion also affected a large range of lipids, including lysophosphocholines, diacyl and acyl ether lipids.

We believe that an absolute decrease in these proposed metabolites (either alone or in combination) is likely to predict deterioration of the disease and which, without intervention would result in disease relapse.

Currently, we are not aware of any studies that demonstrate or state that a decrease in these metabolites in the plasma is likely to predict relapse of IBD.

1. An ex vivo analytical method of determining whether a subject has inflammatory bowel disease (IBD), comprising:
providing an isolated biological sample;
measuring the concentration of at least two biomarkers in the sample;
wherein the concentration is significantly lower or higher than a reference value for the biomarkers indicates that the subject has inflammatory bowel disease,
wherein a first biomarker is a lipid selected from the group consisting of glycerophospholipid, sphingolipid and combinations thereof and at least one further biomarker is an amino acid selected from a group consisting of arginine, glutamine, glycine, histidine, isoleucine, leucine, ornithine, serine, threonine, tryptophane, tyrosine phenylalanine, and valine and combinations thereof.

2. The method of claim 1 wherein the concentration of the biomarkers in the sample significantly lower than a reference value for the biomarkers indicates that the subject has inflammatory bowel disease.

3. The method of claim 1 comprising a phosphatidylcholine selected from the group consisting of monoacylphosphatidylcholine, diacylphosphatidylcholine, and alkylacylphosphatidylcholine.

4. The method of claim 1, wherein at least the concentration of 3 of 5 biomarkers is measured.

5. The method of claim 1, for predicting a relapse of IBD.

6. The method of claim 1, wherein a concentration of the biomarker in the sample of the subject that is significantly lower than a reference value for the biomarker indicates that the subject has increased risk of having an IBD relapse.

7. The method of claim 1, wherein at least one further biomarker is selected from the group of amino acids consisting of arginine, glycine, histidine, serine, tryptophan, and phenylalanine and combinations thereof.

8. The method of claim 7, wherein one further biomarker is arginine and at least one further biomarker is selected from the group of amino acids consisting of glycine, histidine, serine, tryptophan, and phenylalanine and combinations thereof.

9. The method of claim 7, wherein one further biomarker is glycine and at least one further biomarker is selected from the group of amino acids consisting of arginine, histidine, serine, tryptophan, and phenylalanine and combinations thereof.

10. The method of claim 7, wherein one further biomarker is histidine and at least one further biomarker is selected from the group of amino acids consisting of arginine, glycine, serine, tryptophan, and phenylalanine and combinations thereof.

11. The method of claim 7, wherein one further biomarker is serine and at least one further biomarker is selected from the group of amino acids consisting of arginine, glycine, histidine, tryptophan, and phenylalanine and combinations thereof.

12. The method of claim 7, wherein one further biomarker is tryptophan and at least one further biomarker is selected from the group of amino acids consisting of arginine, glycine, histidine, serine, and phenylalanine and combinations thereof.

13. The method of claim 7, wherein one further biomarker is phenylalanine and at least one further biomarker is selected from the group of amino acids consisting of arginine, glycine, histidine, serine, and tryptophan and combinations thereof.

14. The method of claim 7, wherein one further biomarker is selected from the group consisting of the amino acids consisting of arginine, glutamine, isoleucine, serine, and threonine and combinations thereof.

15. The method of claim 1, wherein each biomarker is measured in a separate tissue the sample being selected from the group consisting of whole blood, blood serum, or plasma, tissue of the intestinal wall, tissue of the liver, and mesenteric adipose.

16. The method of claim 1, wherein the patient is selected from the group consisting of a pediatric patient, an adult patient, a patient in a non-acute phase of IBD, and a patient in an acute phase of IBD.

17. The method of claim 1, wherein the concentration of the respective biomarker is lower than the reference value by 10%.

18. Use of the biomarkers according to claim 1 to identify the samples of subjects in need of an IBD intervention.

19. A system for determining whether a subject has inflammatory bowel disease, the system comprising a device for measuring the concentration of the biomarkers through a method comprising: providing an isolated biological sample; measuring the concentration of at least two biomarkers in the sample; wherein the concentration is significantly lower or higher than a reference value for the biomarkers indicates that the subject has inflammatory bowel disease, wherein a first biomarker is a lipid selected from the group consisting of glycerophospholipid, sphingolipid and combinations thereof and at least one further biomarker is an amino acid selected from a group consisting of arginine, glutamine, glycine, histidine, isoleucine, leucine, ornithine, serine, threonine, tryptophane, tyrosine phenylalanine, and valine and combinations thereof, the system comprising a computer:
the computer stores a data base comprising reference values for the concentrations of the biomarkers;
the computer stores a software program having instructions causing the computer
to receive and store the measured values of the parameters of the sample of a subject;
to compare values of said measured parameters to the reference values stored in the system;
indicate the sample as belonging to a subject having inflammatory bowel disease if the measured values differ from the reference values; and
output the results of step iii.

20. A kit for determining whether a subject has inflammatory bowel disease comprising reagents for measuring the concentration of the biomarkers through a method comprising: providing an isolated biological sample; measuring the concentration of at least two biomarkers in the sample; wherein the concentration is significantly lower or higher than a reference value for the biomarkers indicates that the subject has inflammatory bowel disease, wherein a first biomarker is a lipid selected from the group consisting of glycerophospholipid, sphingolipid and combinations thereof and at least one further biomarker is an amino acid selected from a group consisting of arginine, glutamine, glycine, histidine, isoleucine, leucine, ornithine, serine, threonine, tryptophane, tyrosine phenylalanine, and valine and combinations thereof.

21. The kit of claim 20, wherein the reagents are selected from the group of monoclonal antibodies, polyclonal antibodies, single chain antibodies, and F_{c} fragments and wherein the reagents are specific for said biomarkers.
